(19) United States
(12) Patent Application Publication
(10) Pub. No.: US 2020/0239588 A1
(43) Pub. Date: Jul. 30, 2020

(54) METHOD AND COMPOSITION FOR TREATING HEPATOCELLULAR CARCINOMA WITHOUT VIRAL INFECTION BY CONTROLLING THE LIPID HOMEOSTASIS

(71) Applicant: NATIONAL HEALTH RESEARCH INSTITUTES, Zhunan Township, Miaoli County (TW)

(72) Inventors: Shih-Feng TSAI, Zhunan Township, Miaoli County (TW); Yung-Feng LIN, Zhunan Township, Miaoli County (TW); Ning HSU, Zhunan Township, Miaoli County (TW)

(21) Appl. No.: 16/753,670

(22) PCT Filed: Oct. 5, 2018
(86) PCT No.: PCT/US2018/054672

§ 371 Date: Apr. 3, 2020

Related U.S. Application Data
(60) Provisional application No. 62/568,452, filed on Oct. 5, 2017.

(51) Int. Cl. C07K 16/28; A61P 31/14

(52) U.S. Cl. CPC C07K 16/2896; A61K 2039/505; A61P 31/14

(57) ABSTRACT

The present invention relates to a method and a pharmaceutical composition for treating an HCC negative for HBV/HCV, comprising administering a subject in need thereof an therapeutically effective amount of an inhibitory agent to control the genetic alteration of lipid homeostasis associated genes, including CD36 amplification and ABCG4 deletion. According to the present invention, medicines targeting the lipid metabolism pathways are developed to treat HCC patients with CD36 amplification and/or ABCG4 deletion.

### FIELD OF THE INVENTION

The present invention generally relates to a method for treating Hepatocellular carcinoma (HCC) in a subject lacking a history of hepatitis virus infection. In particular, the present invention relates to a method for treating HCC negative for HBV/HCV infection (NBNC-HCC) in a subject by targeting the genetic alteration of lipid homeostasis associated genes, especially by modulating control the CD36 amplification and/or ABCG4 deletion.

### BACKGROUND OF THE INVENTION

Hepatocellular carcinoma (HCC) is a highly malignant tumor of liver. It ranks fifth in the worldwide cancer incidence, causing over 660,000 deaths annually (Jemal, A. et al. C A Cancer J Clin. 57, 43-66, 2007; El-Serag, H. B. N Engl J Med. 365, 1118-1127, 2011). HCC is usually associated with infection by hepatitis B virus (HBV) or hepatitis C virus (HCV) (Farazi, P. A., DePinho, R. A. Nat Rev Cancer 6, 674-687, 2006). However, recently, the incidence of HCC induced by metabolic disorders has been increasing (Starley, B. Q. et al. Hepatology 51, 1820-1832, 2010; Fujiwara, N et al. J Hepatol. S0168-8278, 32328-32340, 2017). The molecular events leading to HCC are not clear in this group of patients who lack a history of viral hepatitis.

Globally, vaccination programs and the development of antiviral medicines have had a significant impact on the management of viral liver diseases and these factors are changing the landscape of liver malignancy. In the US, Japan, Europe, and Australia, the prevalence of nonalcoholic fatty liver disease (NAFLD) is on the rise, and HCC, as a result of nonalcoholic steatohepatitis (NASH), is expected to increase in parallel with the growing epidemics of diabetes and obesity.

Genomic instability is a hallmark of many cancers and this includes HCC (Niu, Z. S et al. World J Gastroenterol. 22, 9069-9095, 2016). Importantly, some cancer-associated genes have been identified and validated by investigating chromosomal instability. However, such studies have not as yet analyzed genomic alterations in relation to the various different risk factors associated with HCC.

The present invention has investigated the clinical and genomic features of HCCs that have different background histories in terms of viral hepatitis infection. Notably, changes in two genes, CD36 and ABCG4, which are linked to lipid homeostasis, have been identified as differentiating markers much frequently detected in HCC that lacks a history of HBV/HCV infection. The information available in public genomic data repository provides independent evidence to support the conclusion that lipid homeostasis genes, comprising CD36 and ABCG4, play a significant role in HCC oncogenesis.

### SUMMARY OF THE INVENTION

In this invention, it is found that consistently CD36 gene amplification is detected in 25% of the HCC samples and more common in the NBNC-HCC samples than in the HBV-HCC samples from the BGI dataset. By investigating the copy number changes in CD36 and ABCG4, CD36 gene amplification and ABCG4 gene deletion are detected in 15.6% and 10.3% of the ICGC HCC samples, respectively, and in 15.3% and 9.7% of the TCGA HCC samples, respectively.

Accordingly, the present invention provides a method for preventing or treating HCC negative for HBV/HCV infection (NBNC-HCC) in a subject, comprising administrating an inhibitory agent to modulatory control the genetic alteration of lipid homeostasis associated genes. In one embodiment of present invention, the genetic alteration comprises CD36 amplification. In another embodiment of present invention, the genetic alteration comprises ABCG4 deletion. In yet another embodiment of present invention, the genetic alteration comprises CD36 amplification and ABCG4 deletion. The modulatory control of CD36 amplification and ABCG4 deletion in HCC is useful to improve a survival rate of the HCC subject.

In another aspect, the present invention provides a composition for preventing or treating HCC negative for HBV/HCV infection (NBNC-HCC) in a subject, comprising a modulatory agent controlling the genetic alteration of lipid homeostasis associated genes. In one embodiment of present invention, the modulatory agent is aimed to suppress CD36 overexpression. In another embodiment of present invention, the modulatory agent is aimed to block lipid uptake in the HCC cells. In an illustrative embodiment of present invention, the modulatory agent is an anti-CD36 antibody.

In one embodiment of present invention, the modulatory agent is aimed to induce ABCG4 expression. In another embodiment of present invention, the modulatory agent is aimed to promote cholesterol transport within the HCC cells. In a preferable embodiment of present invention, the modulatory agent is an ABCG4 protein.

### BRIEF DESCRIPTION OF THE DRAWINGS

The foregoing summary, as well as the following detailed description of the invention, will be better understood when read in conjunction with the appended drawing. In the drawings:

FIG. 1, A and B show the pairwise comparison between the different subtypes of HCC tumors. The DNA copy number changes are analyzed by PennCNV. The statistical analysis of the differences in DNA copy number changes between the HCC subtypes is obtained using Fisher's exact test. (A) The differential pattern of DNA copy number gain between the HCC subtypes. (B) The differential pattern of DNA copy number loss between the HCC subtypes. The significance level is marked using red horizontal lines. The genotype results of the paired HCC samples obtained from the SNP arrays are displayed based on their viral infection history. The PennCNV generates analysis shows (A) DNA copy number gain and (B) DNA copy number loss. The red lines denote significance of P<0.001.

FIG. 2, A-D show CD36 amplification and ABCG4 deletion detected by SNP genotyping array. (A) A DNA copy number gain on chromosome 7. The extent of the DNA amplification affecting the chromosome 7 region is depicted for each tumor. Colors indicate the different subtypes of HCC tumor. Blue: HBV-HCC, green: HCV-HCC, orange: NBNC-HCC. (B) A somatic amplification event affecting the CD36 gene region (Chr7: 80.23 Mb-80.30 Mb) is found to be associated with NBNC-HCC tumors. (C) A DNA copy number loss on chromosome 11. The extent of the DNA deletion affecting the chromosome 11 region is depicted for each tumor. (D) A somatic deletion event within the ABCG4 gene region (Chr11: 119.02 Mb-119.03 Mb) is found to be associated with NBNC-HCC. * P<0.05.

FIG. 3, A-D show the RNA and protein expression of the CD36 gene in NBNC-HCCs. In FIG. 3A, a quantitative PCR is used to measure the CD36 DNA copy number and mRNA expression levels of five pairs of HCC samples that have no history of HBC or HCV infection. The DNA copy number analysis is designed such that it used the 5′ end (5′CN) and 3′ end (3′CN) sequences of the CD36 gene as PCR primers. The results are obtained using triplicate measurements (mean±s.d.). FIG. 3B is the immunohistochemical staining using anti-CD36 antibodies to detect CD36 protein expression in the sample from case 235. The specimen is evaluated under a light microscope at 10× magnification. Further details of specific areas are shown: the tumor tissue section (red box) in FIG. 3C and the non-tumor tissue section (blue box) in FIG. 3D.

FIG. 4 shows the CD36 DNA copy number amplification ratio for 88 HCCs from the BGI dataset. The proportion of cases with CD36 gene amplification is significantly higher in NBNC-HCC tumor tissues as compared with the HBV-HCC tumor tissues. ** P<0.01, Fisher's exact test.

FIG. 5 shows the CD36 gene amplification and ABCG4 gene deletion events obtained from the ICGC and TCGA liver cancer dataset. The proportion of HCC cases positive for the two genetic alterations is similar for the ICGC collection and the TCGA collection.

FIG. 6, A and B show the effects of CD36 gene amplification and ABCG4 gene deletion on HCC patient survival and tumor size. The data analyzed is obtained from the HCC patients whose tumor samples had been genotyped for CD36 amplification and ABCG4 deletion. The median survival rate of patients with both CD36 amplification and ABCG4 deletion in the tumor tissue is significantly shorter than that of the patients with only CD36 amplification or with only ABCG4 deletion (P<0.0001). The statistical analysis is performed using the log-rank test for the data showed in FIG. 6A. In FIG. 6B, the tumor size is significantly increased in HCCs that contain both CD36 amplification and ABCG4 deletion. The error bars indicate standard deviation. Significance levels are indicated as follows: * P<0.05 and ** P<0.01, and are obtained using unpaired Mann-Whitney U test.

FIG. 7, A-D show the effect of anti-CD36 antibody on HCC cell growth. (A) The cell viability of three HCC cell lines is found to be significantly reduced in the presence of 40 μg/ml of anti-CD36 antibodies for 72 hours. (B) The effect of anti-CD36 antibody treatment on HuH-7 cell viability is dose dependent. (C) After undergoing 20 μg/ml anti-CD36 antibody treatment, the cell viability of HuH-7 cells is reduced in a time-dependent manner. (D) The apoptosis ratio of the HuH-7 cells is increased in the presence of 40 μg/ml anti-CD36 antibody as compared to the control antibody (IgG). For all graphs, error bars indicate mean±s.d. Significance levels are indicated as follows: * P<0.05, ** P<0.01 and *** P<0.001, and are obtained using unpaired two-tailed t-tests.

### DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereof known to those skilled in the art.

The present invention provides a method for preventing or treating HCC negative for HBV/HCV infection (NBNC-HCC) in a subject, comprising administrating an inhibitory agent to control the genetic alteration of lipid homeostasis associated genes, especially to affect the CD36 gene amplification and/or ABCG4 gene deletion in the HCC patient.

As used herein, the term “genetic alteration” refers to changes in the normal DNA sequence of a particular gene. When the genetic code is altered, dysfunctional and abnormally activated proteins may be produced to result in cancer. Abnormally activated and dysfunctional proteins may dysregulate normal biologic processes and cells to multiply and become cancerous. Current methods for genome-wide detection of genetic alterations include: molecular cytogenetic evaluation of chromosomal aberrations and re-arrangements; DNA polymorphism analysis for detecting loss of heterozygosity (LOH) or allelic imbalance; and comparative genomic hybridization (CGH) approaches for identifying segmental copy number changes.

As used herein, the term “CD36 gene amplification” refers to an increase in the number of copies of CD36 gene without a proportional increase in other genes. This can result from duplication of a region of DNA that contains the CD36 gene through errors in DNA replication and repair machinery.

As used herein, the term “ABCG4 gene deletion” also called “ABCG4 gene deficiency” or “ABCG4 deletion mutation” refers to a mutation (a genetic aberration) in which a part of a chromosome or a sequence of DNA is lost during DNA replication. Any number of nucleotides can be deleted, from a single base to an entire piece of chromosome. Gene deletion plays a role in birth defects and in the development of cancer.

Pharmaceutical compositions for use in accordance with the present invention may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For parenteral administration, injection is preferred, including intramuscular, intravenous, intraperitoneal, and subcutaneous. For injection, the compositions of the invention may be formulated in aqueous solutions, preferably in physiologically compatible buffers such as Hanks' solution, Ringer's solution, or physiological saline buffer. In addition, the compounds may be formulated in solid form and re-dissolved or suspended immediately prior to use. Lyophilized forms are also included.

For oral administration, the compositions can be formulated by combining the active compounds with pharmaceutically acceptable carriers well known in the art. Such carriers enable the compositions of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

The term “effective amount” refers to a predetermined amount calculated to achieve the desired effect, i.e., to prevent or treat an HCC negative for HBV/HCV infection (NBNC-HCC). In certain embodiments of the invention, the pharmaceutical composition comprises an effective amount of an anti-CD36 antibody for inhibitory control of lipid uptake in the HCC subject with CD36 amplification.

The present invention is further illustrated by the following examples, which are provided for the purpose of demonstration rather than limitation.

### EXAMPLES

The other characteristics and advantages of the present invention will be further illustrated and described in the following examples. The examples described herein are using for illustrations, not for limitations of the invention.

The practice of the present invention will employ technologies comprising conventional techniques of cell biology, cell culture, and genetic engineering, which are within the ordinary skills of the art. Such techniques are explained fully in the literature.

CoreExome-24 SNP Array

Genotyping is performed using the Infinium CoreExome-24 BeadChip (Illumina) to determine the genotypes of the 115 selected pairs of HCC samples and the procedure followed the manufacturer's instructions. Briefly, approximately 200 ng of genomic DNA from either the HCC tumor tissue or the non-tumor tissue of the HCC cases is amplified, fragmented, precipitated, and suspended in hybridization buffer. After denaturation, the samples are hybridized to the BeadChips at 48° C. for 16 hours. Next, single-base extension is performed and the chip is stained and scanned on an Illumina Bead Array Reader. The image dataset is then analyzed using Illumina GenomeStudio v2 software and the standard parameters. The raw genotype data is then output into text files in order to calculate DNA copy number. To detect DNA copy number alterations using the data from the genotyping chips, B allele frequency (BAF) and log R ratio (LRR) are extracted using GenomeStudio v2 software, analyzed by PennCNV v1.0.3 and then visualized by WGAViewer. The validity of the DNA copy number analysis, which is on the SNP array results, is confirmed by carrying out qPCR assays on the HCC samples.

CD36 DNA Copy Number Analysis

To measure the DNA copy number of CD36 in the HCC tumor tissue samples, a quantitative PCR is performed to compare CD36 DNA copy number and the reference DNA copy number between the tumor samples and the adjacent non-tumor tissue. There are designed three primers each to amplify the 5′ end region, the 3′ end region of CD36 gene and a reference sequence. The sequences of the primers used for amplifying the CD36 sequences are as follows: for the 5′ end region: 5′-GGCTCATTCACCAAGGAC (forward, SEQ ID No. 1) and 5′-GACTTAATGAGAAGGAACAAC (reverse, SEQ ID No. 2), for the 3′ end region: 5′-GTTACTACCTTCTCTTCTG (forward, SEQ ID No. 3) and 5′-GTAAAGTGAATCCAGTTATC (reverse, SEQ ID No. 4), for the reference sequence: 5′-GAAACTGTTTTCCTTGTCTG (forward, SEQ ID No. 5) and 5′-GCTTTGTACTGGGAGGAG (reverse, SEQ ID No. 6). All quantitative PCR assays are performed using a SensiFAST™ SYBR® Hi-ROX kit (Bioline) on an ABI StepOne real-time PCR system. The difference in ΔCT between the tumor tissue and adjacent non-tumor tissues is used to calculate CD36 DNA copy number.

Quantitative Analysis of CD36 Expression

The relative expression levels of the tumor (T) tissue and non-tumor (N) tissue samples are determined using reverse transcription quantitative PCR (RT-qPCR). Total RNA from 100 pairs of HCC samples are reverse-transcribed to cDNA using SuperScriptII (Invitrogen) according to the manufacturer's instructions. The subsequent qPCR reactions for the target gene and GAPDH are performed in triplicate on an ABI StepOne real-time PCR system, using a SensiFAST™ SYBR® Hi-ROX kit. The sequences of the primers used for RT-qPCR are as follows: for CD36, 5′-GAACCTATAACTGGATTCAC (forward, SEQ ID No. 7) and 5′-GTCCCAGTCTCATTAAGC (reverse, SEQ ID No. 8) and for GAPDH, 5′-GTGAAGCAGGCGTCGGAG (forward, SEQ ID No. 9) and 5′-GTTGTCATACCAGGAAATG (reverse, SEQ ID No. 10). All samples are analyzed and normalized using the expression level of the internal control gene, GAPDH. The relative quantification in terms of fold-change is obtained by comparing ΔCT of the tumor tissue samples with the ΔCT of the adjacent non-tumor tissue samples.

DNA Copy Number Analysis Using the Public Domain Datasets

Genomic data in the International Cancer Genome Consortium (ICGC) and The Cancer Genome Atlas (TCGA) liver cancer dataset is downloaded from http://xena.ucsc.edu. Whole genome sequencing (WGS) data of 88 paired HCC cases are downloaded from NCBI Sequence Read Archive (SRA) and the accession number is PRJEB2869. To analyze the DNA copy number (CN) changes of CD36 and ABCG4 using the WGS data, the raw reads are aligned to human genome hg19 by Isacc variant caller²⁰ and the DNA copy number are analyzed by CNVSeg.

Statistical Analysis

Mann-Whitney U test is performed to calculate the significance of the clinical data, including age, tumor size, tumor number, and AFP level. Fisher's exact test is used to determine the significance differences of the cirrhosis ratio, fatty liver ratio, and DNA copy number changes between the HCC groups. For anti-CD36 antibody treatment data, two-tailed unpaired Student's t-test is used to compare the magnitude between the different HCC groups or the growth inhibition effect between those treated with anti-CD36 antibody or with control IgG. Survival analysis of the HCC patients is performed by the log-rank test. All data are presented as mean+s.d. and representative three independent experiments. Prism (GraphPad) is used for these analyses.

### Example 1. DNA Copy Number Changes in Chromosome 7 and Chromosome 11 that are Linked to the NBNC-HCC Tumors

Case Selection

In order to identify the genetic features associated with HCC cases that lack a history of viral hepatitis infection, liver samples from 250 HCC cases via the Taiwan Liver Cancer Network (TLCN) are obtained and investigated the genomic profiles of these HCC tissue samples. This biorepository is established with broad consent to support research leading to clinical and pathological information (Chang, I. C. et al. Medicine (Baltimore) 95, e3284, 2016). The study cases include 100 subjects (50 males and 50 females) with a history of HBV infection (HBV-HCC) and 100 subjects (50 males and 50 females) with a history of HCC infection (HCV-HCC). The remaining 50 subjects (25 males and 25 females) with HCC have no prior history of either HBV infection or HCV infection (NBNC-HCC). The clinical features of these HCC cases are presented in Table 1.

TABLE 1
Clinical features of the HCCs with different viral infection histories

| | HBV-HCC | | HCV-HCC | | NBNC-HCC | |
|---|---|---|---|---|---|---|
| | Male | Female | Male | Female | Male | Female |
| Number | 50 | 50 | 50 | 50 | 25 | 25 |
| Age (year) | 54.6¹ | 51.1¹ | 64.9 | 65.3 | 64.8 | 64.9 |
| Tumor size (cm) | 8.64 | 7.38 | 4.22² | 4.31² | 7.76 | 7.85 |
| Tumor number | 3.02 | 1.96 | 1.54 | 1.59 | 1.68 | 1.40 |
| AFP (ng/ml) | 30230³ | 46772³ | 4754 | 3937 | 3879 | 18580⁴ |
| Cirrhosis (%) | 42% | 32% | 46% | 54% | 12%⁵ | 4%⁵ |
| Fatty change | 9/47 | 15/50 | 9/43 | 15/48 | 11/21⁶ | 7/23 |
| (positive/analyzed) | | | | | | |

¹P < 0.0001, comparing onset age for HBV-HCC vs. HCV-HCC or NBNC-HCC
²P < 0.0001, comparing tumor size for HCV-HCC vs. HBV-HCC or NBNC-HCC
³P < 0.05, comparing AFP level for HBV-HCC vs. HCV-HCC or NBNC-HCC
⁴Average increased due to an outlier of unusually high value (380,000)
⁵P < 0.01, comparing cirrhosis percentage for NBNC-HCC vs. HCV-HCC or HBV-HCC
⁶P < 0.01, comparing fatty change percentage for NBNC-HCC vs. HCV-HCC and HBV-HCC

The incidence of cirrhosis is significantly lower in NBNC-HCC group (8%) than in the other two groups (P<0.0001). Furthermore, the male patients in NBNC-HCC group have a significantly higher frequency of individuals with fatty liver change (52.4%) than either the HBV-HCC group (19.1%) or the HCV-HCC group (20.9%).

Genomic Analysis

To conduct genomic analysis, 115 cases are selected based on their TP53 mutation status and these consisted of 38 HBV-HCC, 42 HCV-HCC, and 35 NBNC-HCC individuals (Table 1). To screen for common TP53 hotspot mutations, a detection panel using DNA mass spectrometry is designed. The PCR and extension primers are designed using MassArray Assay Design 3.1 software (Sequenom).

TABLE 2
The TP53 mutation frequencies across the 250 HCC cases

| | Cases | TP53 mutant | TP53 wide type |
|---|---|---|---|
| HBV-HCC | 100 | 20 | 80 |
| HCV-HCC | 100 | 24 | 76 |
| NBNC-HCC | 50 | 13 | 37 |

Genotyping is performed using the Infinium CoreExome-24 BeadChip (Illumina) to determine the genotypes of the 115 selected pairs of HCC samples and the procedure followed the manufacturer's instructions. Briefly, approximately 200 ng of genomic DNA from either the HCC tumor tissue or the non-tumor tissue of the HCC cases is amplified, fragmented, precipitated, and suspended in hybridization buffer. After denaturation, the samples are hybridized to the BeadChips at 48° C. for 16 hours. Next, single-base extension is performed and the chip is stained and scanned on an Illumina Bead Array Reader. The image dataset is then analyzed using Illumina GenomeStudio v2 software and the standard parameters. The raw genotype data is then output into text files in order to calculate DNA copy number. To detect DNA copy number alterations using the data from the genotyping chips, B allele frequency (BAF) and log R ratio (LRR) are extracted using GenomeStudio v2 software, analyzed by PennCNV v1.0.3 and then visualized by WGAViewer. The validity of the DNA copy number analysis, which is on the SNP array results, is confirmed by carrying out qPCR assays on the HCC samples.

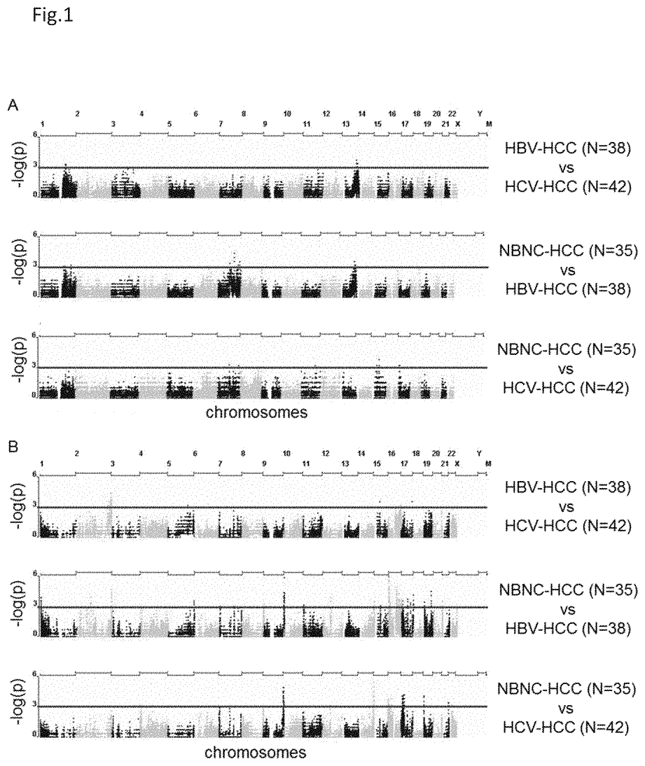

By applying PennCNV software, it is able to identify probes that are able to detect DNA copy changes in the genes of HCC cases. Pair-wise comparisons between the different categories of HCC are performed and showed in FIG. 1. In terms of identifying DNA copy gain, there is a peak on chromosome 13 associated with HBV-HCC individuals and another on chromosome 7 associated with NBNC-HCC individuals (FIG. 1A). In terms of identifying DNA copy loss, there is a peak on chromosome 2 associated with HBV-HCC individuals and another on chromosome 11 associated with NBNC-HCC individuals (FIG. 1B).

Thus, the study concludes that HCC that does not involve HBV/HCV infection shows different clinical manifestations, as well as a distinct chromosomal profile, compared to HCCs associated with HBV/HCV.

### Example 2. CD36 Amplification and ABCG4 Deletion are Frequently Detected in HCC Cases Negative for HBV/HCV Infection

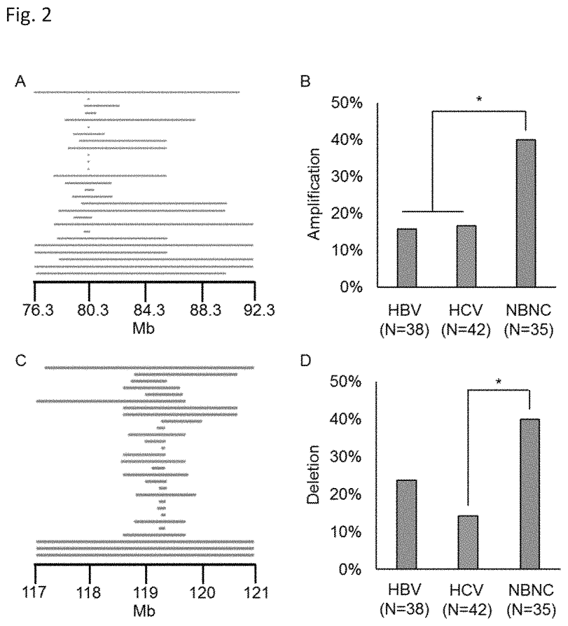

The peaks associated with changes in DNA copy number are further investigated in this example. Since the present invention is most concerned with NBNC-HCC, the region of chromosome 7, an area that extended from 79.1 Mb to 80.7 Mb is analyzed in detail. As shown in FIG. 2A, the probes targeting this region detected DNA copy number gain, and in all positive cases, the amplification involved a specific gene, CD36, at a position near 80.3 Mb. CD36 is an integral membrane protein that functions as a scavenger receptor and it has multiple functions; one of these is to facilitate fatty acid uptake. The analysis of the nature of this DNA amplification event finds that the copy change occurs only in the tumor tissue samples. Moreover, this event is most prominent for the NBNC-HCC individuals (FIG. 2B).

Recent studies have reported that ATP binding cassette (ABC) transporters can play a significant role in the control of cellular and total body lipid homeostasis (Baldan, A. et al. Curr Opin Lipidol. 17, 227-232, 2006). Among these transporters, ABCG1 and ABCG4 are able to form a heterodimer and this heterodimer has been shown to mediate the transport of cholesterol within cells to form lipidated lipoproteins (Hegyi, Z. et al. PLoS One 11, e0156516, 2016). When the gene loci in the region affected by DNA loss on chromosome 11 is searched, it is able to identify somatic deletions that spanned the ABCG4 gene region (FIG. 2C) and loss of ABCG4 occurred in 40% of the NBNC-HCC cases (FIG. 2D). This finding in NBNC-HCC cases, when is integrated with the CD36 gene amplification finding outlined above, supports the hypothesis that genetic alterations involving lipid homeostasis pathways are likely to play a role in the pathogenesis of HCC when there is an absence of a history of viral hepatitis infection.

qPCR Assays

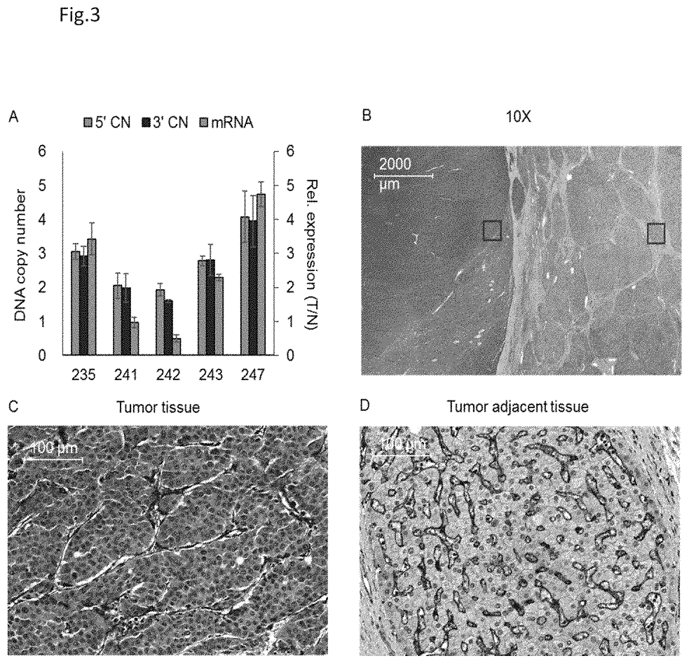

To validate the genotype data obtained from the SNP arrays, qPCR assays are developed to investigate DNA copy number and mRNA expression levels of CD36 in the HCC tissue samples. As shown in FIG. 3A, HCC samples (cases 235, 243, and 247), which had three or more copies of CD36 in the tumor tissue samples, are found to have elevated CD36 mRNA expression levels in the samples, as compared to paired non-tumor tissue samples. Furthermore, CD36 gene expression is elevated in the tumor tissue (T) relative to the non-tumor adjacent tissue (N) in case 235, 243 and 247, each of which has more than three copies of the CD36 gene. On the other hand, in case 241 and 242, the CD36 copy number does not deviate from that predicted for a diploid genome and that the CD36 mRNA expression ratios between the tumor and non-tumor adjacent tissues are close to one. As shown in FIG. 3, anti-CD36 antibodies are able to strongly stain cancer cells (FIG. 3C) in the tumor tissues, which suggest that CD36 protein is overexpressed in the HCC cancer cells. However, by comparison, the CD36 protein is found to be abundantly expressed in the sinusoid endothelial cells, while benign hepatocytes show only very weak CD36 expression (FIG. 3D). The sinusoid endothelial cells in the non-tumor tissue samples are used as an internal positive control.

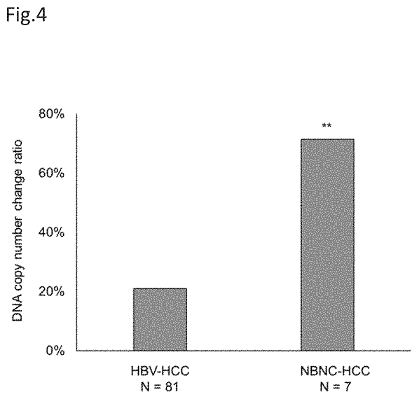

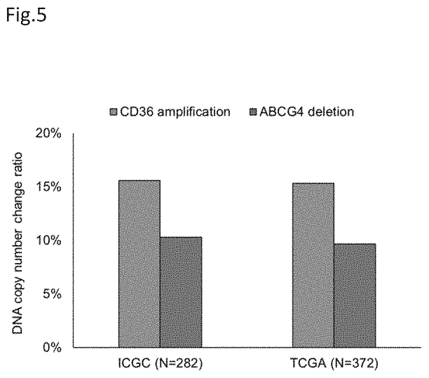

As showed in FIG. 4, consistently CD36 gene amplification is detected in 25% of these HCCs and more common in the NBNC-HCC samples than in the HBV-HCC samples. Next, the genotype data from the ICGC and TCGA databases are investigated and the copy number changes in CD36 and ABCG4 are obtained. As shown in FIG. 5, CD36 gene amplification and ABCG4 gene deletion are detected in 15.6% and 10.3% of the ICGC HCC samples, respectively, and in 15.3% and 9.7% of the TCGA HCC samples, respectively. Thus, the information available in public genomic data repository provides independent evidence that supports our conclusion that these two lipid homeostasis genes play a significant role in HCC oncogenesis.

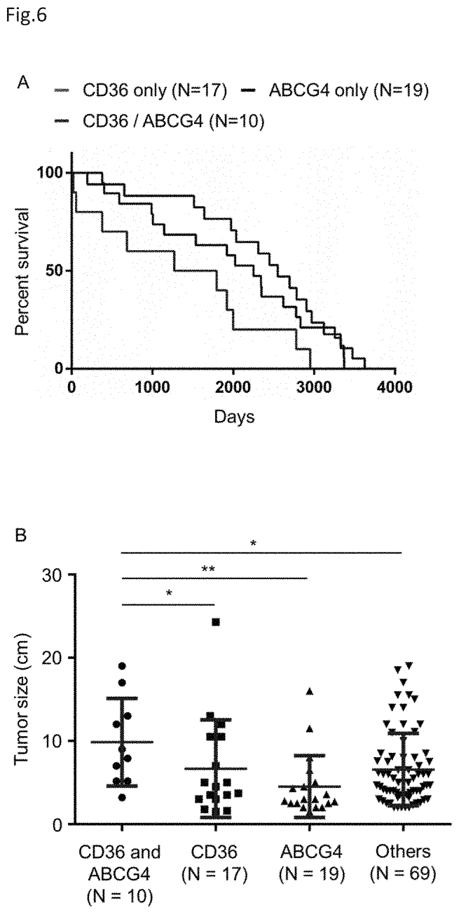

Based on the fact that CD36 is involved in the transport of lipid into hepatocytes, and the report that CD36 plays a role in tumor metastasis (Nath, A. & Chan, C. Sci Rep. 6, 18669, 2016), the impactful effects of CD36 amplification and/or ABCG4 deletion on the clinical features of HCC and on outcome of the patients are further investigated in this example. As shown in FIG. 6A, patients with both CD36 amplification and ABCG4 deletion have a median survival of 1385 days, which is significantly shorter than that of patients with only CD36 amplification (2344 days) or ABCG4 deletion (2045 days) (P<0.0001). Consistently, it is found that CD36 amplification is associated with high α-fetoprotein level (see, Table 3) and that tumor size is significantly increased in HCCs that contained both CD36 amplification and ABCG4 deletion (FIG. 6B).

TABLE 3
The alpha-fetoprotein levels of 115 HCCs grouped
by CD36 amplification and/or ABCG4 deletion

| | Cases | Average | SD | Range |
|---|---|---|---|---|
| CD36 and | 10 | 38799 | 119908 | 1.77-379998 |
| ABCG4 | | | | |
| CD36 | 17 | 19837 | 51049 | 1.81-194153 |
| ABCG4 | 19 | 3911 | 15737 | 2.56-68845 |
| Others | 69 | 7364 | 25649 | 3-173900 |

Genetic alterations that affect CD36 and/or ABCG4 will impact on the lipid homeostasis pathways and this might play a role in HCC oncogenesis. We anticipate that medicines targeting the lipid metabolism pathways should be able to be developed to treat HCC patients with CD36 amplification and/or ABCG4 deletion.

### Example 3. Anti-CD36 Antibodies Inhibited the Growth of HCC Cell Lines

A recent study has reported that CD36 is a marker of metastatic cells. We hypothesize that anti-CD36 antibodies, by blocking lipid uptake in liver cancer cells, would be able to inhibit cell growth. Based on this, experiments using cultured liver cancer cells are carried out in this example.

Cell Proliferation Assay.

The hepatoma cell lines are plated in 96-well plates (10⁴ cells per well) for 24 hours and then treated with DMEM medium containing 3% FBS and anti-CD36 antibodies (Cayman, CAY-188150) for 72 hours. A cell proliferation assay is then performed using alamarBlue cell viability reagent (Thermo Fisher Scientific) according to the user manual.

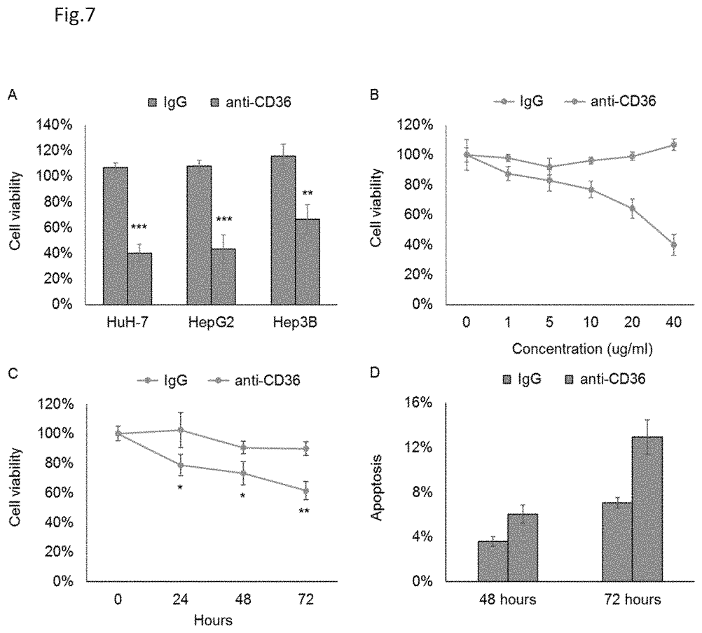

As shown in FIG. 7, the addition of anti-CD36 antibodies at a concentration of 40 g/ml for 72 hours reduces the cell viability of the HuH-7, HepG2, and Hep3B cell lines (FIG. 7A). When using HuH-7 cells, the effect of the anti-CD36 antibodies is found to be concentration dependent (FIG. 7B) and time dependent (FIG. 7C).

Apoptosis Assay.

HuH-7 cells are trypsinized and resuspended in 200 μl ice-cold PBS. For the apoptosis assay, 100 μl of cell suspension is aliquoted into microcentrifuge tubes and harvested by centrifugation at 300 g for 5 min. The cells are then resuspended in 100 μl Annexin V FITC apoptosis detection kit (BD Biosciences) incubation buffer and incubated with 1 μl Annexin V plus 1 μl PI for 15 minutes at room temperature. Before apoptosis detection, 500 μl of incubation buffer are added to cells. The results are obtained using a BD FACSCalibur flow cytometer system (BD Biosciences) that is set to record 5,000 events per sample. The data showed in FIG. 7D also suggests that this effect is probably mediated via an apoptosis-linked mechanism.

In summary, the present invention has investigated the clinical and genomic features of HCCs that have different background histories in terms of viral hepatitis infection. Notably, changes in two genes, CD36 and ABCG4, which are linked to lipid homeostasis, have been identified as differentiating markers more frequently detected in HCC that lacks a history of HBV/HBC infection. Our findings also disclose associations of CD36 amplification and/or ABCG4 deletion with the lowered survival, high α-fetoprotein level and increased tumor size in HCC patients. Therefore, changes in CD36 and ABCG4 genes may provide diagnostic factors for finding and preventing the development of HCC in high-risk individuals.

1. A method for preventing or treating HCC negative for HBV/HCV infection (NBNC-HCC) in a subject, comprising administrating the subject in need thereof a therapeutically effective amount of a modulatory agent to control the genetic alteration of lipid homeostasis associated genes.

2. The method of claim 1, the genetic alteration comprises CD36 amplification.

3. The method of claim 1, wherein the genetic alteration comprises ABCG4 deletion.

4. The method of claim 1, wherein the genetic alteration comprises CD36 amplification and ABCG4 deletion.

5. A composition for preventing or treating HCC negative for HBV/HCV infection (NBNC-HCC) in a subject, comprising a therapeutically effective amount of a modulatory agent for inhibiting or promoting the genetic alteration of lipid homeostasis associated genes.

6. The method of claim 5, wherein the modulatory agent is an agent to suppress CD36 overexpression.

7. The method of claim 5, wherein the modulatory agent is an agent to block lipid uptake in the HCC cells.

8. The method of claim 7, wherein the modulatory agent is an anti-CD36 antibody.

9. The method of claim 5, wherein the modulatory agent is an agent to induce ABCG4 expression.

10. The method of claim 5, wherein the modulatory agent is an agent to promote cholesterol transport within the HCC cells.

11. The method of claim 10, wherein the modulatory agent is an ABCG4 protein.


SEQUENCE LISTING (10 sequences)

SEQ ID NO: 1 - DNA, 18 bp, Artificial sequence
  primer
ggctcattcaccaaggac

SEQ ID NO: 2 - DNA, 21 bp, Artificial sequence
  primer
gacttaatgagaaggaacaac

SEQ ID NO: 3 - DNA, 19 bp, Artificial sequence
  primer
gttactaccttctcttctg

SEQ ID NO: 4 - DNA, 20 bp, Artificial sequence
  primer
gtaaagtgaatccagttatc

SEQ ID NO: 5 - DNA, 20 bp, Artificial sequence
  primer
gaaactgttttccttgtctg

SEQ ID NO: 6 - DNA, 18 bp, Artificial sequence
  primer
gctttgtactgggaggag

SEQ ID NO: 7 - DNA, 20 bp, Artificial sequence
  primer
gaacctataactggattcac

SEQ ID NO: 8 - DNA, 18 bp, Artificial sequence
  primer
gtcccagtctcattaagc

SEQ ID NO: 9 - DNA, 18 bp, Artificial sequence
  primer
gtgaagcaggcgtcggag

SEQ ID NO: 10 - DNA, 19 bp, Artificial sequence
  primer
gttgtcataccaggaaatg